(19) United States
(12) Patent Application Publication
(10) Pub. No.: US 2022/0208395 A1
(43) Pub. Date: Jun. 30, 2022

(54) DRUG INTERFERENCE AND EFFICACY INTELLIGENCE SYSTEM

(71) Applicant: Sai Vaishnavi Pidathala, Tracy, CA (US)

(72) Inventor: Sai Vaishnavi Pidathala, Tracy, CA (US)

(21) Appl. No.: 17/555,399

(22) Filed: Dec. 18, 2021

Related U.S. Application Data
(60) Provisional application No. 63/205,567, filed on Dec. 28, 2020.

(51) Int. Cl. G16H 70/40; G06F 16/903

(52) U.S. Cl. CPC G16H 70/40; G06F 16/90335

(57) ABSTRACT

A Drug Interference and Efficacy Intelligence System (DIEIS) provides a system for users, medical professionals, and researchers for applying knowledge mining, bioinformatics, artificial intelligence, and machine learning methods. The DIEIS advises, recommends, and provides warnings to users and medical professionals about potential risks, side effects and possible options to mitigate adverse effects.

### CROSS-REFERENCE TO RELATED APPLICATIONS

This application claims the benefit of priority under 37 USC § 119 to U.S. Provisional Patent Application Ser. No. 63/205,567, titled: “DRUG INTERFERENCE AND EFFICACY INTELLIGENCE SYSTEM” filed on Dec. 28, 2020, the entire disclosure of which is hereby incorporated by reference for all purposes.

### FIELD OF INVENTION

This application relates to healthcare and patients with comorbidities taking a series of drugs for disease treatment and management. The users of this system include patients, caregivers, healthcare professionals, and researchers.

### BACKGROUND OF THE INVENTION

People often take a variety of drugs without being aware of how different drugs interact with each other. Typically, pharmacies provide instructional and side effect information when dispensing the drugs. However, this information is insufficient because it does not consider the additional medications that the patient may be taking. The information in these pamphlets do not discuss efficacy of the drugs. When treating patients with comorbidities, medical professionals tend to focus only on treatment or medications related to their discipline instead of taking a holistic approach for better medical management of all conditions. They might not be aware of all possible side effects of the variety of drugs a patient may be taking. It is not practical to expect every medical professional to be fully informed of every possible scenario, because each patient presents a unique case. It is not practical to expect every medical professional to be fully informed of every possible scenario, because each patient presents a unique case.

Currently, online websites merely provide very generic drug interference information prescription drugs. This information is not customized for the patient's specific prescriptions and medical conditions particularly for scenarios involving prolonged use of one or more drugs. It is important to understand how effective the treatments are for patients with multiple conditions or comorbidities. As such, there is no system currently that specifically offers information pertaining to efficacy.

### SUMMARY OF THE INVENTION

The claimed invention develops a system for users, medical professionals and researchers applying knowledge mining, bioinformatics, artificial intelligence and machine learning methods. This system is called Drug Interference and Efficacy Intelligence System (DIEIS). Furthermore, the system would advise, recommend and provide warnings to users and medical professionals about potential risks, side effects and possible options to mitigate adverse effects. Such a self-service system does not exist in the current marketplace to help users make informed decisions about the drugs being prescribed to them. This system brings significant value, especially for people living with comorbidities and are taking a series of drugs for the treatment and management of these diseases.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, size, or applicability of the disclosure. It should be noted that for clarity and ease of illustration these drawings are not necessarily made to scale.

FIG. 1 illustrates an embodiment of a flow chart for drug interference and efficacy intelligence with an end-to-end workflow of three identified processes as described herein; and

FIG. 2 illustrates an embodiment of a method for determining the number of pairs of drugs using mathematical expressions as described herein.

### DETAILED DESCRIPTION OF EMBODIMENTS

The following description is presented to enable a person of ordinary skill in the art to make and use embodiments described herein. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the disclosure. Thus, the present disclosure is not intended to be limited to the examples described herein and shown, but is to be accorded the scope consistent with the claims.

It should be understood that the specific order or hierarchy of steps in the process disclosed herein is an example of exemplary approaches. Based upon design preferences, it is understood that the specific order or hierarchy of steps in the processes may be rearranged while remaining within the scope of the present disclosure. Any accompanying method claims present elements of the various steps in a sample order, and are not meant to be limited to the specific order or hierarchy presented.

The present invention puts forth the development of a computer implemented method to provide information on the potential side effects of individual drugs and the potentiality for harmful results of the user taking specific drugs at the same time. The developed system shall provide knowledge to users about side effects and the effective management of drugs for better outcomes.

The system designed will act as a self-help system for users taking one or more drugs on a daily or a periodic basis. The drugs being taken are usually over-the-counter, prescribed by a medical professional, or a combination of each thereof.

The system designed includes an extensive database of information on side effects of individual drugs and their interferences if taken during the same period. The information sources for developing a comprehensive database shall include but not limited to pharmaceutical literatures, research studies, medical journals, public health organizations, academic institutions and firsthand experiences of users and caregivers themselves.

The self-help system can be used by users for “what if” analysis. This means that they don't necessarily have to be taking drugs to understand drug interferences and side effects. The system implements Artificial Intelligence (AI) methods to provide users drug interference and efficacy information based on the derived intelligence from the system's knowledge based on similar drugs.

The system has a provision wherein users such as patients, caregivers, medical professionals, researchers etc. can contribute their inputs to the existing body of knowledge. The system will have a method to validate user offered inputs on side effects of drugs. The method for such a validation is manual or computer implemented.

The system will flag the information presented to a user, thus identifying the source as a common user, medical professional, or research professional etc.

The system provides a feature for the user to specify one or more medical conditions to receive personalized medication management information.

Different use case scenarios for this invention are presented hereafter. All use cases presented herein may not offer complete or accurate information however, they demonstrate the inventive concept in this application and the power of a self-service system.

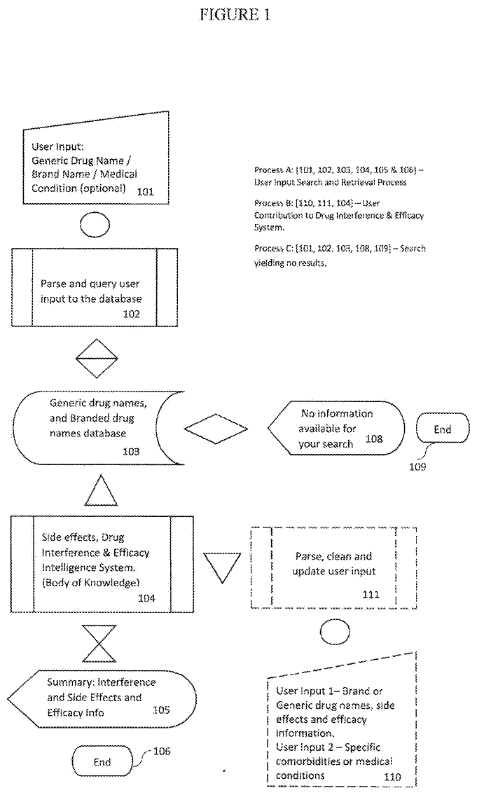

FIG. 1 illustrates an embodiment of a flow chart for determining drug interference and efficacy intelligence with an end-to-end workflow of three identified processes as described herein.

Step 101 illustrates an embodiment of a method for a user to input brand-names or the generic names of one or more drugs and/or specific medical conditions;

Step 102 illustrates an embodiment of a method to parse and identify generic names of individual drugs;

Step 103 illustrates an embodiment of a method where all queried drugs are individually searched for in the database;

Step 104 illustrates an embodiment of a method wherein the knowledgebase is searched to retrieve all information regarding side effects, interference, and efficacy of drugs inputted by the user singularly or in pairs;

Step 105 illustrates an embodiment of a method wherein all retrieved information from Step 104 is displayed to a user with appropriate flags based on the source of information;

Step 106 illustrates an embodiment of a method wherein a search for drug names not found in the database is displayed accordingly to the user as no results found;

Step 110 illustrates an embodiment of a method wherein a user can contribute information about the side effects of individual drugs, effective management of drugs, and warnings regarding drug interactions between pairs of drugs to the system;

Step 111 illustrates an embodiment of a method wherein user input to the system's body of knowledge is processed and updated;

Steps 101, 102, 103, 104, 105 & 106 involved in Process Ain FIG. 1 demonstrate the process of the user seeking information regarding Drug Interference and Efficacy Intelligence.

Steps 110, 111, &104 involved in Process B in FIG. 1 demonstrate the process of the user contributing to the Drug Interference and Efficacy Intelligence System's knowledgebase.

Steps 101, 102, 103, 108, & 109 involved in Process C in FIG. 1 demonstrate the process when no matches are found for the inputted drugs.

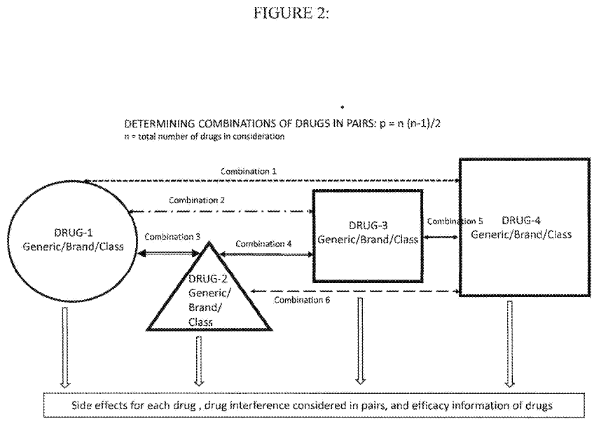

FIG. 2 illustrates an embodiment of a method for determining the number of pairs of drugs using mathematical expressions as described herein. As shown in this figure, the combination of Drugs-1, 2, 3, and 4 are considered in terms of the side effects of each drug, drug interference considered in pairs, and efficacy information for each drug.

Use Case 1

The user inputs Tamoxifen and BCG seeking information from the system.

Sample Search Results:

Important! Seek advice from a medical professional.

Chemo drugs are immunosuppressants.

BCG is a biologic with inactivated TB bacteria given to activate human immune response for fighting cancer cells. Therefore, seek advice if it is safe to be administered while taking chemo drugs [Flagged: Common User].

Extreme weakness for first two days after the administration of BCG into the bladder, barely could get up from the bed for restroom [Flagged: Common User].

From the results above, it can be noted that the user is presented with marketplace info as well as the user provided feedback on drugs.

The use case stated above is an example how the system produces drug interference intelligence to the user.

Use Case 2

The user inputs drugs: Syndopa and Pantocid DSR

Sample Search Results:

B12 levels needs to be checked periodically if taking Pantocid DSR for long periods of time.

Some PPI class drugs increase the risk of drug induced Parkinson's disease. Pantocid DSR helps with the efficacy of Parkinson drug Syndopa by reducing amount of Helicobacter Pylori bacteria in the gut which interferes with the drug performance. Seek the advice of a medical professional if taking these drugs simultaneously. [Flagged: Medical Professional].

Reduced levels of stomach acid for longer periods could reduce immunity and lead to certain types of infection [Flagged: Common User]

The use case stated above is an example of how the system delivers drug efficacy information to the user.

Use Case 3

The user inputs branded drug: Imuran, prescribed for paraneoplastic dermatomyositis with breast cancer.

Sample Search Results:

Watch out for elevated levels of SGOT, a liver enzyme, so frequent blood tests may be required. [Flagged: Common User] [Flagged: Medical Professional]

This use case presents an example of how the system provides information regarding long term side effects of a single drug.

Use Case 4

The user inputs branded drug: Cystopen

Sample Search Results:

Take 3 capsules on empty stomach and expect slow results.

The drug reduces interaction of chemicals and irritants to the bladder lining and thus promoting growth and strengthening of the lining. [Flagged: New England Journal of Medicine]

Dosage less than 3 capsules is generally ineffective. However, some patients may not tolerate 2 or 3 capsules a day. Start with 1 per day, and increase dosage up to 3 a day depending on tolerance. [Flagged: Medical Professional]

Expect to take drug for at least 3 months to see a better outcome or any improvement [Flagged: Common User].

The use case stated above is an example how the system delivery drug efficacy information to the user.

While the inventive features have been particularly shown and described with reference to preferred embodiments thereof, it will be understood by those in the art that the foregoing and other changes may be made therein without departing from the spirit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

What is claimed is:

1. A computer implemented method of determining drug interference and efficacy intelligence for a combination of generic and/or brand-named drugs, comprising:
inputting one or more generic and/or brand-named drugs;
searching a database for matches of the two or more generic and/or brand-named drugs;
retrieving side effect information for each of the two or more generic and/or brand-named drugs;
determining a number of pairs of the generic and/or brand-named drugs based on a total number of the generic and/or brand-named drugs inputted;
considering each pair of the generic and/or brand-named drugs one at a time;
retrieving drug interference information using a proprietary algorithm for all pairs of the generic and/or brand-named drugs;
retrieving efficacy information using a proprietary algorithm for each of the generic and/or brand-named drugs inputted; and
summarizing the side effects, drug interference, and efficacy information for each of the pairs of the generic and/or brand-named drugs.

2. The computer implemented method of claim 1 wherein a database containing information regarding side effects of drugs and interference is maintained.

3. The computer implemented method of claim 1 further comprising allowing a user to input drugs by brand-names and/or generic names.

4. The computer implemented method of claim 1 wherein a database of brand-names and generic names of drugs are maintained.

5. The computer implemented method of claim 1 further comprising allowing a user to input information regarding side effects of long-term use of individual drugs.

6. The computer implemented method of claim 1 further comprising allowing a user to input information regarding drug interference.

7. The computer implemented method of claim 1 further comprising allowing a user to input efficacy information of specific drugs.

8. The computer implemented method of claim 1 further comprising allowing a user to input specific comorbidities and/or medical conditions.

9. The computer implemented method of claim 1 wherein flagging sources of information in the display summary is based on the category of user who provided the inputs to the system.

10. The computer implemented method of claim 1 wherein the number of pairs of drugs to be considered for drug interference intelligence is determined by the number of drugs inputted by a user.

11. The computer implemented method of claim 1 wherein a user is notified if brand-names and/or generic names of drugs inputted by a user yields no results within the database.
